(19) United States
(12) Patent Application Publication
(10) Pub. No.: US 2021/0023565 A1
(43) Pub. Date: Jan. 28, 2021

(54) REACTION APPARATUS AND TEMPERATURE CONTROL METHOD

(71) Applicant: FUJIFILM Corporation, Tokyo (JP)

(72) Inventors: Yoshihisa USAMI, Kanagawa (JP); Yasuyuki ISHII, Kanagawa (JP); Yuki INOUE, Kanagawa (JP); Aya OUCHI, Kanagawa (JP)

(73) Assignee: FUJIFILM Corporation, Tokyo (JP)

(21) Appl. No.: 17/035,370

(22) Filed: Sep. 28, 2020

Related U.S. Application Data
(63) Continuation of application No. PCT/JP2018/047104, filed on Dec. 20, 2018.

(30) Foreign Application Priority Data
Mar. 29, 2018 (JP) 2018-063899

(51) Int. Cl. B01L 7/00; C12Q 1/686

(52) U.S. Cl. CPC B01L 7/52; C12Q 1/686; B01L 2300/0663; B01L 2300/1894; B01L 2300/1811; B01L 2300/1822

(57) ABSTRACT

The reaction apparatus includes a cell in which a specimen liquid of 1 μL or more is accommodated, a cooling plate structure that encloses the cell and consists of a cooling plate, a cooling unit that cools the cooling plate structure, and a heating unit that irradiates at least one of the cell or the specimen liquid with electromagnetic waves, in which the cooling plate configuring the cooling plate structure is in contact with at least two main surfaces of the cell, and the cooling plate structure includes a transparent window that transmits the electromagnetic waves.

### CROSS-REFERENCE TO RELATED APPLICATIONS

This application is a continuation application of International Application No. PCT/JP2018/047104, filed Dec. 20, 2018, the disclosure of which is incorporated herein by reference in its entirety. Further, this application claims priority from Japanese Patent Application No. 2018-063899, filed Mar. 29, 2018, the disclosure of which is incorporated herein by reference in its entirety.

### BACKGROUND OF THE INVENTION

### 1. Field of the Invention

The present disclosure relates to a reaction apparatus and a temperature control method using the reaction apparatus.

### 2. Description of the Related Art

In recent years, various technological developments relating to gene diagnosis have been advanced. The technology of gene diagnosis is used in a wide range of fields such as analysis of disease-related genes in the medical field, microbial inspection and genetically modified crops test in the food field, and paternity test in forensic medicine.

In the gene diagnosis technology, a polymerase chain reaction (PCR) is widely used as technology for amplifying a small amount of deoxyribonucleic acid (DNA).

The DNA amplification by PCR is realized by repeating a step of dissociating double-stranded DNA into single-stranded DNA at a high temperature (a heat denaturation step), a step of lowering the temperature thereafter to bond the primer to the single-stranded DNA (an annealing step), and a step of newly synthesizing double-stranded DNA by polymerase using single-stranded DNA as template (an extension step). One cycle of 94° C. for 1 minute, 50° C. to 60° C. for 1 minute, 72° C. for 1 to 5 minutes, which is repeated 20 to 30 times is exemplified as an example of a temperature cycle.

In PCR, it is necessary to repeatedly raise and lower the temperature of the sample as described above. The time required for raising and lowering the temperature greatly contributes to the PCR time, and how to raise and lower the temperature in a short time is a major point in shortening the PCR. The technology for shortening the temperature cycle for PCR has been proposed in WO2004/029241A, JP2012-125262A, JP2009-542213A, and Analytical Chemistry 2001, Vol. 73, No. 16, p. 4037-4044.

In WO2004/029241A, a reaction apparatus has been proposed in which the temperature can be rapidly raised by locally heating the periphery of the sample with electromagnetic induction heating, and since a reaction container other than the portion containing the material capable of generating heat with electromagnetic induction heating is not heated, the temperature can be rapidly lowered.

JP2012-125262A discloses a configuration in which a heating heater is brought into contact with a heat transfer block on which the reaction container is placed, a cooling apparatus is comprised so as to be movable between a position in contact with the heater and a separated position apart from the heater, and the cooling apparatus is brought into contact with the heater and the heat transfer block is cooled via the heater in a case of lowering the temperature.

JP2009-542213A discloses a device including a chip having a microfluidic channel, an electromagnetic energy source such as laser light, and an energy absorbing element that absorbs the electromagnetic wave and transfers heat to a sample. Further, a configuration is disclosed in which a heat exchange channel is comprised adjacent to the microfluidic channel, and in a case of cooling, the radiation by the electromagnetic energy source is stopped and the cooling fluid is taken into the heat exchange channel for cooling.

Analytical Chemistry 2001, Vol. 73, No. 16, p. 4037-4044 discloses a configuration in which a sample in a channel is irradiated with infrared light to heat the sample, the channel is arranged on a Peltier stage, and cooled by a Peltier element. According to the apparatus in Analytical Chemistry 2001, Vol. 73, No. 16, p. 4037-4044, it is disclosed that a heating rate of 67° C./second and a cooling rate of 53° C./second are possible for a sample of 5 nL.

### SUMMARY OF THE INVENTION

However, in the reaction apparatus disclosed in WO2004/029241A, the heat spreads to a portion other than the heating body heated with electromagnetic induction, resulting in energy loss, and the rate of temperature lowering cannot be said to be sufficient.

The apparatus disclosed in JP2012-125262A needs to comprise a moving mechanism for moving the cooling apparatus, which causes a problem that the device becomes large and is easily broken.

The apparatus disclosed in JP2009-542213A has a configuration in which the insulating fluid in the heat exchange channel is exchanged with the cooling fluid during cooling, and the rate of temperature lowering is still insufficient.

In apparatus disclosed in Analytical Chemistry 2001, Vol. 73, No. 16, p. 4037-4044, since a very small amount of specimen liquid of 5 nL is a target, it can perform heating and cooling at a very high speed. However, in order to be subjected to various analyzes after PCR, it is desirable to target at least about 1 μL of the specimen liquid, and cooling rate decreases in a case where the specimen liquid of 1 μL or more is a target using the method of Analytical Chemistry 2001, Vol. 73, No. 16, p. 4037-4044.

As described above, in the PCR in the related art, the cooling time is rate-determining and the shortening of the reaction time is insufficient.

The present disclosure has been made in view of the above circumstances, and an object of the present disclosure is to provide a reaction apparatus and a temperature control method capable of realizing raising and lowering the temperature rapidly.

A reaction apparatus according to an aspect of the present disclosure comprises a cell in which 1 μL or more of a specimen liquid is accommodated, a cooling plate structure that encloses the cell and comprises a cooling plate, a cooling unit that cools the cooling plate structure, and a heating unit that irradiates at least one of the cell or the specimen liquid with electromagnetic waves, in which the cooling plate configuring the cooling plate structure is in contact with at least two main surfaces of the cell, and the cooling plate structure includes a transparent window that transmits the electromagnetic waves.

In the reaction apparatus according to the aspect of the present disclosure, it is preferable that the cooling unit comprise a Peltier element.

In the reaction apparatus according to the aspect of the present disclosure, it is preferable that the heating unit comprise a light source that emits light having a wavelength of from 0.5 μm to 1.4 μm as the electromagnetic waves.

In the reaction apparatus according to the aspect of the present disclosure, it is preferable to further comprise a control unit that controls irradiation of the electromagnetic waves by the heating unit. The control unit may control cooling by the cooling unit.

In the reaction apparatus according to the aspect of the present disclosure, it is preferable that the control unit performs control such that the specimen liquid is heated to a first temperature by the heating unit in a state in which a temperature of the cooling plate structure is maintained by the cooling unit at a second temperature that is at least 20° C. lower than the first temperature.

The reaction apparatus according to the aspect of the present disclosure may further comprise a temperature sensor that detects a temperature of the specimen liquid.

In the reaction apparatus according to the aspect of the present disclosure, it is preferable that an absorbing body that absorbs energy of the electromagnetic waves and generates heat be mixed into a portion of the cell that is in contact with the specimen liquid.

A temperature control method according to another aspect of the present disclosure is a temperature control method in the reaction apparatus according to the aspect of the present disclosure, comprising performing a temperature control in which a heating step of heating the specimen liquid to a first temperature, and a cooling step of cooling the specimen liquid to a second temperature that is at least 20° C. lower than the first temperature, are repeated, while a temperature of the cooling plate structure is maintained at the second temperature by the cooling unit.

In the temperature control method according to the aspect of the present disclosure, the second temperature may be at least 30° C. lower than the first temperature.

In the temperature control method according to the aspect of the present disclosure, in the heating step, irradiation of the electromagnetic waves by the heating unit may be performed, and in the cooling step, irradiation of the electromagnetic waves by the heating unit may be stopped.

In the temperature control method according to the aspect of the present disclosure, before the heating step and the cooling step, an absorbing body that absorbs energy of the electromagnetic waves and generates heat may be mixed into the specimen liquid accommodated in the cell.

A reaction apparatus according to an aspect of the present disclosure comprises a cell in which a specimen liquid of 1 μL or more is accommodated, a cooling plate structure that encloses the cell and consists of a cooling plate, a cooling unit that cools the cooling plate structure, and a heating unit that irradiates at least one of the cell or the specimen liquid with electromagnetic waves. The cooling plate configuring the cooling plate structure is in contact with at least two main surfaces of the cell, and the cooling plate structure includes a transparent window that transmits the electromagnetic waves. The temperature of the specimen liquid can be rapidly raised by emitting the electromagnetic waves through the transparent window, and since the cell accommodating the specimen liquid is included in the cooling plate structure, the temperature of the specimen liquid can be rapidly lowered.

### BRIEF DESCRIPTION OF THE DRAWINGS

FIG. 1 is a diagram showing a schematic configuration of a reaction apparatus according to an embodiment of the present embodiment.

FIG. 2 is a plan view of a cooling plate structure comprised in the reaction apparatus shown in FIG. 1.

FIG. 3 is an exploded perspective view of a cell comprised in the reaction apparatus shown in FIG. 1.

FIG. 4 is a diagram showing a chart of a temperature control.

### DESCRIPTION OF THE PREFERRED EMBODIMENTS

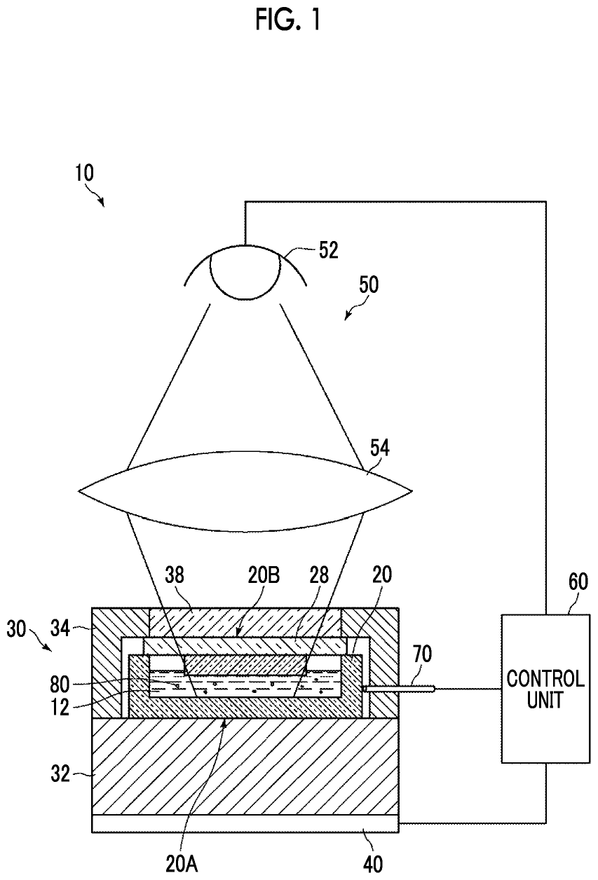

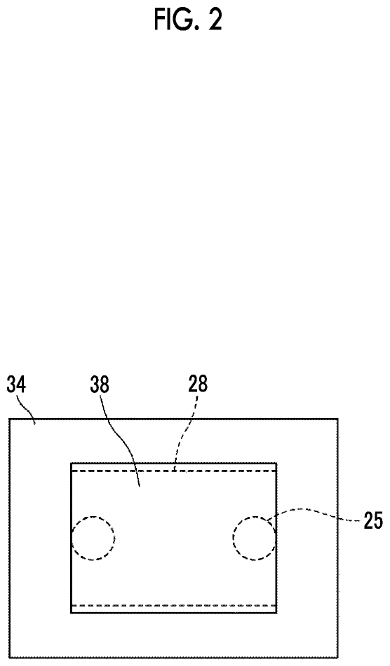

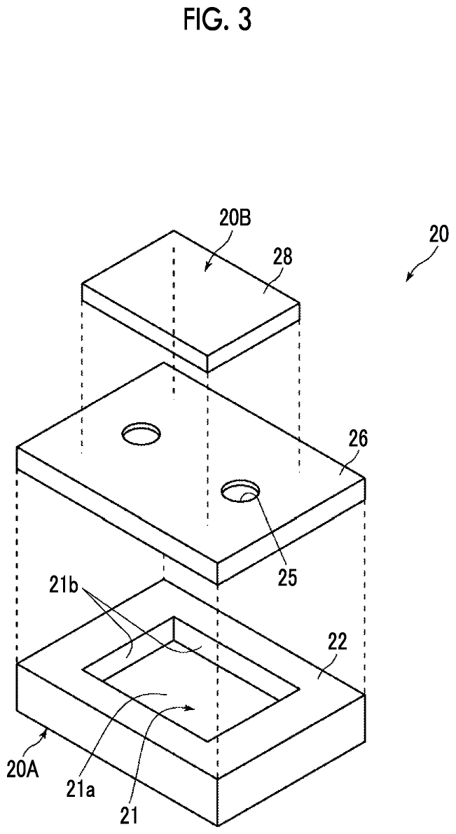

Hereinafter, embodiments of the present invention will be described in detail with reference to the drawings. FIG. 1 is a diagram showing a schematic configuration of a reaction apparatus according to an embodiment of the present embodiment. FIG. 2 is a plan view of a cooling plate structure of the reaction apparatus, and FIG. 3 is an exploded perspective view of a cell comprised in the reaction apparatus.

A reaction apparatus 10 according to the embodiment of the present invention comprises a cell 20 in which a specimen liquid 12 of 1 μL or more is accommodated, a cooling plate structure 30 that encloses the cell 20 and consists of a cooling plate, a cooling unit 40 that cools the cooling plate structure 30, and a heating unit 50 that irradiates at least one of the cell 20 or the specimen liquid 12 with electromagnetic waves. Furthermore, the reaction apparatus 10 comprises a cooling unit 40 and a control unit 60 that controls the heating unit 50.

The reaction apparatus 10 performs a heating process and/or a cooling process on the specimen liquid 12 in order to promote a desired reaction in the specimen liquid 12 accommodated in the cell 20.

As shown in FIG. 3, the cell 20 includes an accommodating unit 21 that accommodates the specimen liquid 12. The accommodating unit 21 has a capacity of 1 μL or more. The capacity of the accommodating unit 21 is preferably 5 μL or more, and more preferably 10 μL or more. In addition, the capacity of the accommodating unit 21 is preferably 1 mL or less, more preferably 500 μL or less, still more preferably 100 μL or less.

The cell 20 comprises a main body 22 having the recessed accommodating unit 21 that accommodates the specimen liquid of 1 μL or more, and a lid 26 that is installed on the main body 22 so as to cover the accommodating unit 21. In a case of use, the main body 22 and the lid 26 are welded or adhered to each other at the periphery to be integrated.

The lid 26 includes two openings 25 facing the accommodating unit 21 of the main body 22. The opening 25 functions as an injection hole and/or a discharge hole for the specimen liquid 12, or an air hole in a case of injecting and discharging the specimen liquid.

The cell 20 comprises a sealing film 28 for sealing the opening 25. After the specimen liquid 12 is injected into the accommodating unit 21 through opening 25, the sealing film 28 is attached to the surface of the lid 26 in order to prevent the evaporation of the specimen liquid and the contamination of dust, and the cell 20 is installed in the reaction apparatus 10 in the above state.

In the cell 20 installed on the reaction apparatus 10, a bottom surface 20A of the cell 20 is a bottom surface of the main body 22, and a top surface 20B of the cell 20 is a front surface of the sealing film 28.

The main body 22 and the lid 26 of the cell 20 may be made of any material as long as the material does not react with the specimen liquid 12, and may be any of plastic, ceramic, or metal, or a combination thereof. However, it is necessary that at least a part thereof is made of a material that transmits electromagnetic waves emitted from the heating unit. Examples of the materials that transmit electromagnetic waves include acrylic, polystyrene (PS), polycarbonate (PC), polypropylene (PP), polyethylene terephthalate (PET), polyvinyl chloride (PVC), polyester (PE), polyamide (PA), polyimide (PI), an acrylonitrile-butadiene-styrene copolymer (ABS), and polylactic acid (PLA). In particular, PS, PC, PP, PET, and PVC are preferable. In addition, a portion contacting the heat conductive plate may be made of a high heat conductive member. Specifically, it is plastic or metal. As the metal, aluminum, copper, iron, and an alloy containing at least one of aluminum, copper, or iron are preferable.

As the sealing film 28, a resin film that does not react with the specimen liquid 12 and transmits electromagnetic waves can be used.

It is preferable that an absorbing body that absorbs the energy of the irradiated electromagnetic waves and generates heat be mixed in a portion of the cell 20 which is in contact with the specimen liquid 12, specifically, at least a part of inner wall surfaces 21a and 21b configuring the accommodating unit 21 of the cell 20. By comprising such an absorbing body, the heat absorbing amount of the cell due to irradiation with electromagnetic waves increases, so that the speed of raising the temperature can be increased.

Examples of the absorbing bodies include coloring materials such as black paint, and metals such as iron, cobalt, aluminum, copper, and platinum. As a specific color material, for example, 6× Loading Buffer Double Dye (model number 313-90351) or 6×Loading Buffer Orange G (model number 317-90251) manufactured by Nippon Gene Co., Ltd. can be used.

An absorbing body 80 may be dispersed in the specimen liquid 12 instead of or in addition to mixing the absorbing body in the inner wall surfaces 21a and 21b of the accommodating unit 21 of the cell 20. In the specimen liquid 12, the absorbing body 80 may be floating or may be attached to the inner wall surfaces 21a and 21b of the cell 20. The absorbing body to be dispersed in the specimen liquid 12 need only be made of a material having low reactivity with the specimen liquid 12, and the same material as the absorbing body material mixed with the cell 20 described above can be used. A particulate absorbing body (hereinafter referred to as “absorption particles”) may be added to the specimen liquid, and as the absorption particles, metal particles, polymer particles, metal nanoparticles and carbon nanotubes can also be used.

The cooling plate structure 30 is a structure configured by a cooling plate and having a space for accommodating the cell 20 formed therein. In the present embodiment, a plurality of cooling plates 32, 34, and 38 configures the cooling plate structure 30. The cooling plate 38 functions as a transparent window that transmits the electromagnetic waves emitted from the heating unit (hereinafter, also referred to as a transparent window 38).

The cooling plates 32, 34, and 38 are made of a material having high thermal conductivity. In particularly, metal or glass is preferable. As the metal, aluminum, copper, iron, and an alloy containing any of aluminum, copper, and iron are preferable. The transparent window 38 need only transmit a wavelength of the electromagnetic wave emitted from the heating unit 50, and in a case where the heating unit 50 emits infrared light, for example, the transparent window 38 need only transmit infrared light. Glass having a light-transmitting property is suitable as the transparent window 38. In particular, it is preferable that the transparent window 38 be made of glass, and other portions be made of metal plates.

The cooling plate structure 30 is cooled by the cooling unit 40 described below. It is preferable that a portion (the cooling plate 32 in the present embodiment) directly cooled by the cooling unit 40 be a metal block having a large heat capacity. The heat capacity of the cooling plate structure is at least larger than the heat capacity of the cell. The heat capacity of the cooling plate structure is more preferably larger than the heat capacity of the cell by one digit or more.

The cooling plate configuring the cooling plate structure 30 is in contact with at least two main surfaces of the cell 20. The two main surfaces include at least a surface having the largest area among the surfaces forming the outer shape of the cell 20. In the present embodiment, as shown in FIG. 1, the bottom surface 20A of the cell 20 is in contact with the cooling plate 32 of the cooling plate structure 30, and the top surface 20B of the cell 20 is in contact with the cooling plate 38. In the present embodiment, the bottom surface 20A is a surface having the largest area among the surfaces configuring the outer shape of the cell 20, and the top surface 20B is a surface having the second large area. Since two main surfaces 20A and 20B of the cell 20 are in contact with the cooling plate, it is possible to obtain a high cooling effect.

In the present embodiment, the cooling unit 40 is configured by a Peltier element, and is disposed in contact with one cooling plate 32 among the cooling plates 32, 34, and 38 configuring the cooling plate structure 30. Since the cooling plate 32 and the other cooling plates 34 and 38 are in contact with each other directly or indirectly, and are made of a material having a high thermal conductivity, almost entire cooling plate structure 30 has a uniform temperature.

The configuration element of the cooling unit 40 is not limited to a Peltier element as long as a temperature of the cooling plate structure 30 is maintained at a desired temperature, and may be an air cooling mechanism such as a fan and a liquid cooling mechanism using water or other liquids. From the viewpoint of miniaturization, a Peltier element is preferable. Also, different cooling mechanisms may be used in combination. For example, a radiating fin may be provided on the back surface of the Peltier element (the surface opposite to the surface in contact with the cooling plate structure) and a fan that sends air to the radiating fin may be provided.

The heating unit 50 is means for raising the temperature of the specimen liquid 12, and includes an electromagnetic wave source 52. The heating unit 50 may directly heat the specimen liquid 12, may indirectly heat the specimen liquid 12 by heating the cell accommodating the specimen liquid 12, or may directly and indirectly heat the specimen liquid 12.

The electromagnetic wave emitted by the heating unit 50 preferably has a wavelength of 0.1 μm or more and 1000 μm or less. The electromagnetic wave preferably has a wavelength of 0.2 μm or more and 100 μm or less, more preferably 0.4 μm or more and 200 μm or less, and particularly preferably light having a wavelength of 0.5 μm or more and 1.4 μm or less in the visible light to infrared light region.

As the electromagnetic wave source 52, a filament lamp such as a halogen lamp, a light emitting diode (LED) light source, a laser light source, or the like can be used.

In the present embodiment, the heating unit 50 comprises a condensing optical system 54 that condenses the electromagnetic waves emitted from the electromagnetic wave source 52. Since the electromagnetic waves emitted from the electromagnetic wave source 52 can be condensed and irradiated on the specimen liquid 12 and the periphery (the cell 20) of the specimen liquid 12 due to the condensing optical system 54, it is preferable to comprise the condensing optical system 54.

The control unit 60 controls at least the irradiation with electromagnetic waves by the heating unit 50. In the present embodiment, the same control unit 60 is configured to also control the cooling by the cooling unit 40. The cooling unit 40 may be controlled by a separate control unit. The control unit 60 is configured by, for example, a computer including a central processing unit (CPU), a semiconductor memory, and a hard disk. A temperature control program is installed in the hard disk of the control unit 60, and executes the temperature control.

The temperature control by the control unit 60 may be an analog method or a digital method. The control unit 60 may perform proportional control, integral control, differential control, or a proportional-integral-differential (PID) control in which proportional control, integral control, and differential control are combined.

In the reaction apparatus 10, in a case where the temperature control is performed in which a heating step of heating the specimen liquid to a first temperature and a cooling step of cooling the specimen liquid to a second temperature lower than the first temperature by 20° C. are repeated, the control unit 60 performs a control in which the specimen liquid 12 is heated to the first temperature by the heating unit 50 in a state where a temperature of the cooling plate structure 30 is maintained at the second temperature lower than the first temperature by 20° C. or more by the cooling unit 40. That is, since the heating unit 50 heats the specimen liquid locally while the cooling unit 40 maintains the temperature of the cooling plate structure 30 at the second temperature, in a case where the heating by the heating unit 50 is stopped, the specimen liquid 12 is rapidly cooled to the temperature of the cooling plate structure 30, that is, to the second temperature. For example, the control unit 60 may perform a control to maintain the temperature of the cooling plate structure 30 at a constant temperature by PID control, with respect to the cooling unit 40, and may perform a control in which the electromagnetic wave irradiation is turned on only in a case of heating and the electromagnetic wave irradiation is turned off in a case of temperature lowering, with respect to the heating unit 50.

The reaction apparatus 10 may comprise a temperature sensor 70 that measures a temperature of the specimen liquid 12. In the present embodiment, the reaction apparatus 10 comprises a thermocouple as the temperature sensor 70, and a tip of the thermocouple is disposed in contact with the cell 20. In a case where a correlation between the temperature of the cell 20 and the temperature of the specimen liquid 12 in the reaction apparatus 10 is measured in advance, it is possible to measure the temperature of the specimen liquid 12 indirectly by measuring the temperature of the cell 20.

As the temperature sensor 70, a non-contact-type sensor such as an infrared sensor may be used instead of a contact-type sensor such as a thermocouple. The reaction apparatus may comprise a plurality of temperature sensors for measuring the temperature of the specimen liquid at different locations. Furthermore, the reaction apparatus may comprise the temperature sensor for measuring the temperature of the cooling plate structure 30. Each temperature sensor may be connected to the control unit 60 and used for feedback control during temperature control.

The reaction apparatus 10 according to the present embodiment comprises the heating unit 50 that emits the electromagnetic waves, and the cooling plate structure 30 comprises the transparent window 38 that transmits the electromagnetic waves from heating unit 50, and thus the temperature of the specimen liquid 12 of 1 μL or more can be raised rapidly by emitting the electromagnetic waves. In the reaction apparatus 10, since the cell 20 accommodating the specimen liquid 12 is included in the cooling plate structure 30, and the bottom surface 20A and the top surface 20B of the cell 20 are in contact with the cooling plate, the temperature of the specimen liquid 12 of 1 μL or more can be lowered rapidly as compared with a case where the only one surface of the cell 20 is in contact with the cooling plate.

By using the reaction apparatus 10 according to the present embodiment, it is possible to execute a process of repeatedly heating and cooling the specimen liquid of 1 μL or more with a temperature difference of 20° C. or more, preferably 30° C. or more, and thus DNA amplification by the PCR method can be executed in a very short time. In a case where the specimen liquid is 1 μL or more, it can be applied to various analyzes such as analysis by immunochromatography, fluorescence detection method, and analysis by electrophoresis.

Next, PCR using an embodiment of the temperature control method of the present invention will be described.

The specimen liquid 12 is prepared. The specimen liquid 12 is, for example, a PCR reaction liquid prepared by extracting DNA from a biological sample such as collected blood, saliva and hair root, or a sample such as a crop, purifying the DNA, and adding a PCR reagent containing a primer to the DNA.

The specimen liquid 12 is injected into the accommodating unit 21 through the opening 25 of the lid 26 of the cell 20. Thereafter, the opening 25 is sealed with the sealing film 28 of the cell 20, and the cell 20 is set in the cooling plate structure 30. At this time, the bottom surface 20A of the cell 20 is set in contact with the cooling plate 32 and the top surface 20B of the cell 20 is set in contact with the cooling plate 38 (the transparent window 38).

In PCR, the DNA amplification is realized by repeating a heat denaturation step of dissociating double-stranded DNA contained in the specimen liquid into single-stranded DNA, an annealing step of bonding the primer to the single-stranded DNA, and an extension reaction step of newly synthesizing double-stranded DNA by polymerase.

For example, the heat denaturation occurs at a temperature of 94° C. to 98° C., the annealing occurs at a temperature of 55° C. to 65° C., and the extension reaction occurs at a temperature of 70° C. to 75° C. However, in order to shorten the time, it is often possible to combine the annealing step and the extension reaction step into one step. That is, DNA amplification can be performed by repeating raising the temperature of the specimen liquid 12 to the first temperature that causes heat denaturation and lowering the temperature to the second temperature that causes annealing and extension reaction.

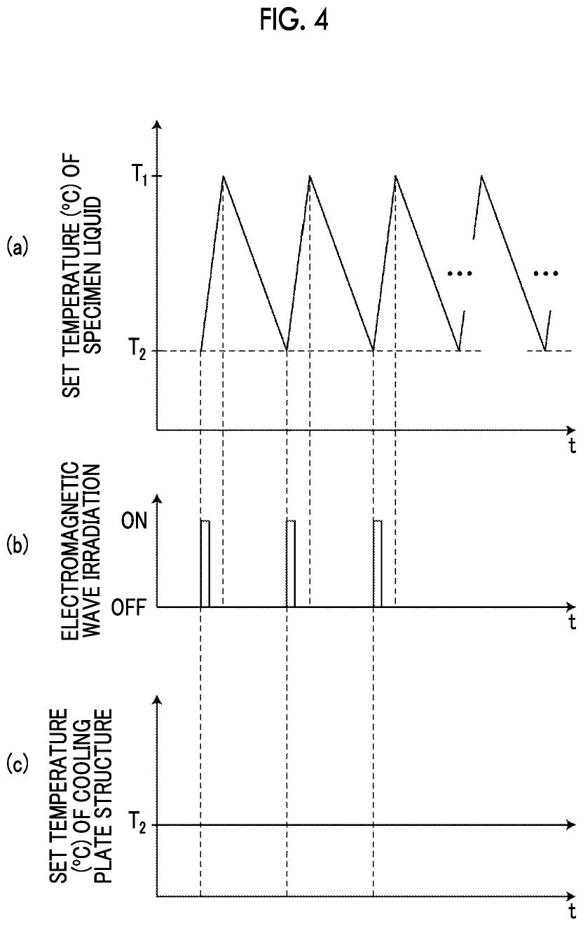

A temperature control method of repeating the first temperature and the second temperature will be described with reference to FIG. 4. (a) of FIG. 4 is a chart showing a set temperature of the specimen liquid, a vertical axis indicates a set temperature (° C.) of the specimen liquid, and a lateral axis indicates time. (b) of FIG. 4 is a timing chart of an on-and-off control of the electromagnetic wave source 52 in the heating unit 50. (c) of FIG. 4 is a chart showing a set temperature of the cooling plate structure 30 cooled by the cooling unit 40, a vertical axis indicates a set temperature (° C.) of the cooling plate structure, and a lateral axis indicates time.

In a case where the temperature control is performed in which heating of raising the temperature of the specimen liquid 12 to a first temperature T₁ and cooling of lowering the temperature to a second temperature T₂ are repeated as shown in (a) of FIG. 4 as an example of the temperature raising and lowering history, the control is performed, with respect to the heating unit 50, in which the electromagnetic wave irradiation is turned on (lighted) at the start of heating and the electromagnetic wave irradiation is turned off (lights-out) after a predetermined time as shown in (b) of FIG. 4. Since the temperature of the specimen liquid 12 increases rapidly by the irradiation with the electromagnetic wave, the irradiation time of the electromagnetic wave is set shorter than the temperature rising time. The relationship between the irradiation time and the temperature rising time to the first temperature T₁ and the temperature lowering time from the first temperature T₁ to the second temperature T₂ is measured in advance, and in a case of actual reaction, the irradiation time and an irradiation time interval need only be set based on the relationship measured in advance.

On the other hand, as shown in (c) of FIG. 4, the cooling unit 40 is controlled so that the temperature of the cooling plate structure 30 is always maintained at the second temperature T₂. The control may be proportional control, integral control, differential control, or PID control.

Example of the temperature control in PCR includes a control of repeating a heating process in which the specimen liquid is held at 45° C. for 1 minute, heated and held at 95° C. for 1 minute, and then heated to 98° C. which is the first temperature T₁, and a cooling process in which the specimen liquid is cooled to 60° C. which is the second temperature T₂ 40 times. In the reaction apparatus 10, the temperature rising time from 60° C. to 98° C. can be, for example, 1 second, and the temperature rising time from 98° C. to 60° C. can be, for example, 6 seconds. The first temperature T₁ is a temperature that causes heat denaturation, and the second temperature T₂ is a temperature that causes annealing and extension reaction. In the example, the difference between the first temperature T₁ and the second temperature T₂ is 38° C.

As described above, only the temperature of the specimen liquid and the cell around the specimen liquid is locally raised to the first temperature while the temperature of the cooling plate structure 30 is maintained at the second temperature, and thus the specimen liquid 12 can be rapidly cooled, and the temperature lowering time can be shortened. As a result, the time required for the PCR reaction can be shortened and various test results can be acquired faster.

The disclosure of JP2018-063899A filed on Mar. 29, 2018 is incorporated herein by reference in its entirety. All documents, patent applications, and technical standards mentioned herein are incorporated herein by reference to the same extent as a case where each individual document, patent application, and technical standard are specifically and individually noted to be incorporated by reference.

What is claimed is:

1. A reaction apparatus, comprising:
a cell in which 1 μL or more of a specimen liquid is accommodated;
a cooling plate structure that encloses the cell and comprises a cooling plate;
a cooling unit that cools the cooling plate structure;
a heating unit that irradiates at least one of the cell or the specimen liquid with electromagnetic waves; and
a control unit that controls irradiation of the electromagnetic waves by the heating unit, wherein:
the cooling plate configuring the cooling plate structure is in contact with at least two main surfaces of the cell,
the cooling plate structure includes a transparent window that transmits the electromagnetic waves,
the control unit controls cooling by the cooling unit, and
the control unit performs control such that the specimen liquid is heated to a first temperature by the heating unit in a state in which a temperature of the cooling plate structure is maintained by the cooling unit at a second temperature that is at least 20° C. lower than the first temperature.

2. The reaction apparatus according to claim 1, wherein the cooling unit comprises a Peltier element.

3. The reaction apparatus according to claim 1, wherein the heating unit comprises a light source that emits light having a wavelength of from 0.5 μm to 1.4 μm as the electromagnetic waves.

4. The reaction apparatus according to claim 1, further comprising a temperature sensor that detects a temperature of the specimen liquid.

5. The reaction apparatus according to claim 1, wherein an absorbing body that absorbs energy of the electromagnetic waves and generates heat is mixed into a portion of the cell that is in contact with the specimen liquid.

6. A temperature control method in the reaction apparatus according to claim 1, the method comprising:
performing a temperature control in which a heating step of heating the specimen liquid to a first temperature, and a cooling step of cooling the specimen liquid to a second temperature that is at least 20° C. lower than the first temperature, are repeated, while a temperature of the cooling plate structure is maintained at the second temperature by the cooling unit.

7. The temperature control method according to claim 6, wherein the second temperature is at least 30° C. lower than the first temperature.

8. The temperature control method according to claim 6, wherein:
the heating step includes performing irradiation of the electromagnetic waves by the heating unit, and
the cooling step includes stopping irradiation of the electromagnetic waves by the heating unit.

9. The temperature control method according to claim 6, further comprising, before the heating step and the cooling step, mixing an absorbing body that absorbs energy of the electromagnetic waves and generates heat into the specimen liquid accommodated in the cell.
